(19) United States
(12) Patent Application Publication
(10) Pub. No.: US 2005/0289665 A1
(43) Pub. Date: Dec. 29, 2005

(54) Method for disinfecting seed

(76) Inventor: Jeffrey E. Hartle, Tacoma, WA (US)

Correspondence Address: WEYERHAEUSER COMPANY;INTELLECTUAL PROPERTY DEPT., CH 1J27, P.O. BOX 9777, FEDERAL WAY, WA, 98063, US

(21) Appl. No.: 11/094,881

(22) Filed: Mar. 30, 2005

Related U.S. Application Data
(60) Provisional application No. 60/582,412, filed on Jun. 24, 2004.

(51) Int. Cl. A01N 25/26; A01H 1/00; C12N 15/82; A01H 7/00

(52) U.S. Cl. 800/278; 800/319; 504/100

(57) ABSTRACT

The invention provides methods for disinfecting seeds, for example, conifer seeds such as loblolly pine and Douglas-fir. The methods comprise the step of contacting seed with an effective concentration of a disinfecting agent comprising at least one of o-phenylphenol and o-benzyl-p-chlorophenol.

### CROSS REFERENCE TO RELATED APPLICATION

The present application claims the benefit of U.S. Provisional Application No. 60/582,412, filed Jun. 24, 2004.

### FIELD OF THE INVENTION

The invention is directed to methods for disinfecting natural seed, particularly conifer seed.

### BACKGROUND OF THE INVENTION

It is often desirable to plant large numbers of genetically identical plants that have been selected to have advantageous properties, but in many cases it is not feasible to produce such plants using standard breeding techniques. In vitro culture of somatic or zygotic plant embryos can be used to produce large numbers of genetically identical embryos that have the capacity to develop into normal plants. One method of producing somatic embryos is to induce “cleavage polyembryogeny” of zygotic embryos, which involves dissecting zygotic embryos from seeds. In order to avoid contamination, seeds are disinfected before the dissection of embryos. Conventional methods for disinfecting seeds involve the use of hazardous and expensive chemicals such as hydrogen peroxide or mercuric chloride. There is a need for reliable and inexpensive methods for disinfecting seed that minimize exposure to harmful chemicals and that do not have an adverse effect on germination. The present invention addresses this and other needs.

### SUMMARY OF THE INVENTION

In one aspect, the invention provides methods for disinfecting seed. The methods comprise the step of contacting seed with an effective concentration of a disinfecting agent comprising at least one of o-phenylphenol and o-benzyl-p-chlorophenol. Concentrations of o-phenylphenol that are useful in the methods of the invention are between about 0.1% and about 0.7%. Concentrations of o-benzyl-p-chlorophenol that are useful in the methods of the invention are between about 0.05% and about 0.5%. In some embodiments, the disinfecting agent comprises both o-phenylphenol and o-benzyl-p-chlorophenol. In some embodiments of the invention, seed is contacted with a disinfecting agent for between about 5 minutes and about 30 minutes, such as between about 10 minutes and about 20 minutes.

The methods of the invention may be used with seed from any plant species. For example, the methods of the invention may be used with conifer seed, such as loblolly pine seed or Douglas-fir seed.

Another aspect of the invention provides methods for obtaining sterile conifer embryos for somatic embryogenesis. The methods comprise the steps of: (a) contacting a conifer seed with an effective concentration of a disinfecting agent comprising at least one of o-phenylphenol and o-benzyl-p-chlorophenol; and (b) removing an embryo from the contacted conifer seed under sterile conditions.

The methods of the invention are useful for providing disinfected seed prior to germination and for providing a sterile source of embryogenic tissue for somatic embryogenesis.

### DETAILED DESCRIPTION OF THE PREFERRED EMBODIMENT

Unless specifically defined herein, all terms used herein have the same meaning as they would to one skilled in the art of the present invention.

Unless stated otherwise, all concentration values that are expressed as percentages are weight per volume percentages.

The invention provides methods for disinfecting seed to allow sterile dissection of the embryo in the seed. The methods comprise the step of contacting seeds with an effective concentration of a disinfecting agent comprising at least one of o-phenylphenol and o-benzyl-p-chlorophenol. The term “effective concentration of a disinfecting agent” refers to an amount of disinfecting agent effective to kill all or most microbial contaminants on the seeds. The methods of the invention are applicable to seeds from any plant species, including conifers. For example, the seed may be a conifer seed, such as a Douglas-fir seed or a loblolly pine seed.

In some embodiments of the invention, the seed is contacted with an effective concentration of o-phenylphenol, as described in EXAMPLES 2 and 4-6. Effective concentrations of o-phenylphenol may be between about 0.1% and about 1%, such as between about 0.2% and about 0.7%. The seed may be contacted with o-phenylphenol for between about 5 minutes and about 30 minutes. For example, seed may be contacted with between about 0.1% to about 1% o-phenylphenol for about 15 minutes, as described in EXAMPLE 2.

In some embodiments of the invention, the seed is contacted with an effective concentration o-benzyl-p-chlorophenol, as described in EXAMPLES 2 and 4-6. Effective concentrations of o-benzyl-p-chlorophenol may be between about 0.01% and about 0.5%, such as between about 0.2% and about 0.4%. The seed may be contacted with o-benzyl-p-chlorophenol for between about 10 minutes and about 30 minutes. For example, seed may be contacted with between about 0.05% to about 0.5% o-benzyl-p-chlorophenol for about 15 minutes, as described in EXAMPLE 2.

In some embodiments of the invention, the seed is contacted with effective concentrations of both o-phenylphenol and o-benzyl-p-chlorophenol, as described in EXAMPLES 2 and 4-6. For example, seed may be contacted with between about 1% to about 10% Amphyll (Lysol I.C., 10.5% o-phenylphenol, 5% o-benzyl-p-chlorophenol, 1.8% isopropyl alcohol, National Laboratories, New Jersey) for about 15 minutes.

In some embodiments of the invention, the seed is contacted with an effective concentration of sodium hypochlorite, as described in EXAMPLES 1, 2, and 7, in addition to contacting seed with an effective concentration of at least one o-phenylphenol and o-benzyl-p-chlorophenol. For example, seed may be contacted with an effective concentration of sodium hypochlorite before or after contacting seed with an effective concentration of at least one o-phenylphenol and o-benzyl-p-chlorophenol. Effective concentrations of sodium hypochlorite may be between about 0.5% and about 3.5%. The seed may be contacted with sodium hypochlorite for between about 5 minutes and about 30 minutes. For example, seed may be contacted with about 2.6% sodium hypochlorite for about 15 minutes.

In the methods of the invention, seed may first be washed with a surfactant before contacting seed with an effective concentration of the disinfecting agent in order to enhance the penetration of the disinfecting agent. For example, the seed may be washed with a detergent, such as a dishwashing detergent, as described in EXAMPLES 1-7. Other suitable surfactants that may be used in accordance with the methods of the invention include, but are not limited to, Tween (such as Tween-20 or Tween-80), dishwashing soap, and non-toxic wetting agents.

The methods of the invention provide disinfected seed without damaging the embryo or affecting germination. Thus, the methods of the invention allow the sterile dissection of the embryo within the seed for use as a source of embryogenic tissue in somatic embryogenesis. Moreover, the methods of the invention are as effective in terms of absence of contamination and normalcy of germinants as methods using more expensive and more hazardous disinfecting agents such as mercuric chloride or hydrogen peroxide, as shown in EXAMPLES 1 and 4-6. The term “normalcy of germinants” denotes the presence of all expected parts of a plant at the time of evaluation.

Another aspect of the invention provides methods for obtaining sterile conifer embryos for somatic embryogenesis. The methods comprise the steps of: (a) contacting a conifer seed with an effective concentration of a disinfecting agent comprising at least one of o-phenylphenol and o-benzyl-p-chlorophenol; and (b) removing an embryo from the contacted conifer seed under sterile conditions. The concentrations of disinfecting agents and the duration of the disinfecting treatments useful in the practice of this aspect of the invention are as described above. Exemplary methods for obtaining sterile conifer seed are described in EXAMPLES 2-5.

A further aspect of the invention provides methods for initiating the formation of conifer embryogenic cells. The methods comprise the steps of: (a) contacting a conifer seed with an effective concentration of a disinfecting agent comprising at least one of o-phenylphenol and o-benzyl-p-chlorophenol; (b) removing an embryo from the contacted conifer seed under sterile conditions; and (c) culturing the embryo in an induction medium to initiate the formation of embryogenic cells. The concentrations of disinfecting agents and the duration of the disinfecting treatments useful in the practice of step (a) of this aspect of the invention are as described above. Exemplary methods for obtaining sterile conifer seed according to step (b) are described in EXAMPLES 2-5.

This aspect of the invention is generally useful for the production of conifer somatic embryos. Methods for producing conifer somatic embryos from conifer zygotic embryos have been previously described (see, e.g., U.S. Pat. Nos. 4,957,866; 5,034,326; 5,036,007; 5,041,382; 5,236,841; 5,294,549; 5,482,857; 5,563,061; and 5,821,126, all of which are incorporated herein by reference). Typically, zygotic embryos are cultured in an induction medium to initiate the formation of embryogenic cells, such as embryonic suspensor masses that are capable of developing into somatic embryos. The embryogenic cells may then be further cultured in a maintenance medium that promotes establishment and multiplication of the embryogenic cells. Subsequently, the multiplied embryogenic cells may be cultured in a development medium that promotes the development of somatic embryos, which may further be subjected to post-development treatments.

In step (c) of the methods of this aspect of the invention, an embryo is cultured in an induction medium to initiate the formation of embryogenic cells. Induction media generally contain plant nutrients and hormones such as auxins and/or cytokinins. Any induction medium that effectively initiates the formation of conifer embryogenic cells is suitable for use in step (c) of the methods. Exemplary induction media are described, for example, in U.S. Pat. Nos. 4,957,866; 5,041,382; 5,236,841; 5,294,549; 5,482,857; 5,563,061; and 5,821,126.

The following examples merely illustrate the best mode now contemplated for practicing the invention, but should not be construed to limit the invention.

### EXAMPLES

### Example 1

This Example compares the use of mercuric chloride and sodium hypochlorite in disinfection treatments for Douglas-fir seed on contamination and normalcy of germinants.

Methods: Douglas-fir seeds were glued to a Petri plate with super glue (25 seeds/plate) and allowed to sit overnight. The next day, the plates containing the seed were disinfected using one of two methods. The first treatment used the following steps at room temperature, unless otherwise indicated:
(1) Each plate was filled with nanopure water and left for one hour;
(2) The water was removed and each plate was filled with a 10% solution of Liquinox (VWR Scientific) and stirred on a culture shaker at 10 rpm for 1 hour;
(3) The Liquinox was removed and each plate was rinsed four times with nanopure water for 10 minutes each;
(4) In a sterile hood, each plate was filled with 0.5% HgCl₂ and left for 15 minutes;
(5) The HgCl₂ was removed and each plate was rinsed three times with nanopure water for 10 minutes each;
(6) Each plate was filled with 30% H₂O₂ (Sigma Chemical Co.) and left for 15 minutes;
(7) The H₂O₂ was removed and each plate was rinsed 13 times with sterile nanopure water for 20 minutes each; and
(8) Each plate was filled with sterile nanopure water, covered with parafilm, and kept at 4° C. overnight.

The second treatment was identical to the first treatment except that 50% Clorox brand bleach (5.25% sodium hypochlorite) was used instead of HgCl₂ in step (4). Embryos were dissected out of seed using sterile technique and placed on Petri plates containing medium 185 (Table 1).

TABLE 1
Composition of Medium 185

| Constituent | NM2 (mg/l) |
|---|---|
| NH₄NO₃ | 206.25 |
| KNO₃ | 1170 |
| MgSO₄.7H₂O | 185 |
| KH₂PO₄ | 85 |
| CaCl₂.2H₂O | 220 |
| KI | 0.415 |
| H₃BO₃ | 3.1 |
| MnSO₄.H₂O | 8.45 |
| ZnSO₄.7H₂O | 4.3 |
| Na₂MoO₄.2H₂O | 0.125 |
| CuSO₄.5H₂O | 0.0125 |
| CoCl₂.6H₂O | 0.0125 |
| FeSO₄.7H₂O | 13.925 |
| Na₂EDTA | 18.625 |
| Nicotinic acid | 0.5 |
| Pyridoxine.HCl | 0.5 |
| Thiamine.HCl | 1 |
| Glycine | 2 |
| Myo-Inositol | 100 |
| Sucrose | 20 |
| Agar | 18 |
| Charcoal | 2.5 |
| | pH adjusted |
| | to 5.7 |

Results: Both treatments worked equally well in terms of absence of contamination and normalcy of germinants.

### Example 2

This Example compares the use of four different concentrations of a disinfectant containing o-phenylphenol and o-benzyl-p-chlorophenol in disinfection treatments for Douglas-fir seed on contamination and normalcy of germinants.

Methods: Douglas-fir seeds were glued to a Petri plate with super glue (25 seeds/plate) and allowed to sit overnight. The next day, the plates containing the seed were disinfected using one of four treatments. The first treatment used the following steps at room temperature, unless otherwise indicated:
(1) Each plate was filled with nanopure water and left for one hour;
(2) The water was removed and each plate was filled with a 10% solution of Liquinox (VWR Scientific) and stirred on a culture shaker at 10 rpm for 1 hour;
(3) The Liquinox was removed and each plate was rinsed four times with nanopure water for 10 minutes each;
(4) In a sterile hood, each plate was filled with 50% Clorox brand bleach (5.25% sodium hypochlorite) and left for 15 minutes;
(5) The Clorox was removed and each plate was rinsed three times with nanopure water for 10 minutes each;
(6) Each plate was filled with 1% Amphyll (Lysol I.C., 10.5% o-phenylphenol, 5% o-benzyl-p-chlorophenol, 1.8% isopropyl alcohol, National Laboratories, New Jersey) and left for 15 minutes;
(7) The Amphyll was removed and each plate was rinsed 3 times with sterile nanopure water for 20 minutes each; and
(8) Each plate was filled with sterile nanopure water, covered with parafilm, and kept at 4° C. overnight.

Treatments 2-4 were identical to the first treatment except that the concentration of Amphyll used was 2.5% in treatment 2, 5% in treatment 3, and 10% in treatment 4. After disinfection and hydration, seed were removed from the tea strainers and placed on a sterile Petri plate. Seed were individually removed with sterile forceps, cracked using autoclaved Vice-Grips, and placed in sterile quadrant Petri plates (2 seeds/quadrant) containing a drop of sterile water to avoid dehydration before dissection. After dissection, 50 whole seed and 50 dissected embryos were incubated in TSYG broth (Trypticase Soy Broth 30 g/l, Yeast Extract 5 g/l, Glucose 10 μl, and—for a solid Petri plate or agar slant—Agar 15 g/l) and examined on a daily basis for signs of contamination during an eight week incubation period at 30° C.

Results: All four treatments worked equally well in terms of absence of contamination and normalcy of germinants, as shown in Table 2.

TABLE 2
Normalcy, Organ Lengths, and Contamination at Day 44

| | | Radicle | | | Contami- |
|---|---|---|---|---|---|
| Treat- | Normalcy | Length | Hypocotyl | Cotyledon | nation |
| ment | (%) | (cm) | Length (cm) | Length (cm) | (%) |
| 1 | 98 | 9.22 | 1.95 | 0.83 | 0 |
| 2 | 100 | 9.17 | 1.82 | 0.71* | 0 |
| 3 | 100 | 8.95 | 1.86 | 0.76 | 0 |
| 4 | 98 | 7.54 | 1.86 | 0.76 | 0 |

*indicates significant difference.

### Example 3

This Example tests the effect of hydrogen peroxide in disinfection treatments for loblolly pine seed on contamination of whole seed and dissected embryos.

Methods: The following disinfection treatment was used for loblolly pine seed:
(1) Seed was placed in a tea strainer (about 100 seed/strainer) and soaked in deionized water for one hour with agitation;
(2) The seed in the tea strainer was soaked in a 10% solution of Liquinox (VWR Scientific) for one hour with agitation;
(3) The seed in the tea strainer was soaked in 30% H₂O₂ on a rotary shaker in a fume hood for 15 minutes;
(4) The seed in the tea strainer was rinsed 8 times for 10 minutes each in sterile water in a laminar flow hood;
(5) The seed in the tea strainer was hydrated in sterile water for 24 hours in a laminar flow hood; and
(6) The seed in the tea strainer was rinsed three times with sterile water for 10 minutes each.

After disinfection and hydration, seed were removed from the tea strainers and placed on a sterile Petri plate. Seed were individually removed with surface-sterilized latex gloves, cracked using autoclaved Vice-Grips, and placed in sterile quadrant Petri plates (2 seeds/quadrant) containing a drop of sterile water to avoid dehydration before dissection. After dissection, 50 whole seed and 50 dissected embryos were incubated in TSYG broth (Trypticase Soy Broth 30 g/l, Yeast Extract 5 g/l, Glucose 10 g/l, and—for a solid Petri plate or agar slant—Agar 15 g/l) and examined on a daily basis for signs of contamination during an eight week incubation period at 30° C.

Results: There was no contamination of dissected embryos. By the third day of incubation, contamination was present in 4 out of 50 whole seed (8%). After eight weeks of incubation, 68% of the whole seed was contaminated. It is known that it is almost impossible to sterilize seed without damaging the embryo. The goal is to remove as many microbes from the seedcoat as possible to permit sterile dissection of the embryo from the megagametophyte.

### Example 4

This Example compares the use of hydrogen peroxide and a disinfectant containing o-phenylphenol and o-benzyl-p-chlorophenol in disinfection treatments for loblolly pine seed on contamination and germination of dissected embryos.

Methods: The first treatment used the following steps at room temperature, unless otherwise indicated:
(1) Seed was placed in a tea strainer and soaked in ultra pure water for one hour with agitation;
(2) The seed in the tea strainer was soaked in a 10% solution of Liquinox (VWR Scientific) for one hour with agitation;
(3) The seed was rinsed with running ultra pure water until no foam was apparent;
(4) The tea strainers were transferred to a sterilization chamber and 30% hydrogen peroxide was added to the chamber, a vacuum of 15-20 inches of mercury was pulled on the sterilizer and released three times, and the seed was allowed to soak in the hydrogen peroxide for 15 minutes;
(5) The seed was rinsed four times for 20 minutes each with filter-sterilized de-ionized water;
(6) The seed was removed from the tea strainer and placed in a sterile one-liter beaker, sterile ultra pure water was added to the beaker until the strainers were submerged, the beaker was covered with foil and placed at 5° C. overnight.

The second treatment was identical to the first except that no vacuum was applied to the sterilizer. The third treatment was identical to the first except that seed was pre-imbibed with water for 24 hours before the first step. The fourth treatment was identical to the third except that no vacuum was applied to the sterilizer. The fifth treatment was identical to the first except that in step (4) the tea strainers were transferred to a sterile beaker to which 2.5% v/v Amphyll was added, the air bubbles removed by tapping the tea strainer in the beaker, and the seed allowed to soak for 15 minutes. Embryos were dissected from seed subjected to treatments 1-5 and from untreated seed.

After disinfection and hydration, seed were removed from the tea strainers and placed on a sterile Petri plate. Seed were individually removed with sterile forceps, cracked using autoclaved Vice-Grips, and placed in sterile quadrant Petri plates (2 seeds/quadrant) containing a drop of sterile water to avoid dehydration before dissection. After dissection, 50 whole seed and 50 dissected embryos were incubated in TSYG broth (Trypticase Soy Broth 30 g/l, Yeast Extract 5 g/l, Glucose 10 g/l, and—for a solid Petri plate or agar slant—Agar 15 g/l) and examined on a daily basis for signs of contamination during an eight week incubation period at 30° C.

Results: There was no contamination of dissected embryos from any of the five treatments. Thus, the use of Amphyll is as effective as hydrogen peroxide as a disinfecting agent. It is apparent that pre-imbibition of seed before disinfection is detrimental to germination because hypocotyl length, percent normalcy, and the percentage of malformed embryos were significantly worse after treatments 3 and 4, as shown in Tables 3 and 4. Provided the embryo is dissected out of seed within 24 hours, it develops to the same degree as an embryo dissected out of an untreated seed.

TABLE 3
Organ Lengths at Day 24 After Dissection

| | Radicle Length | Hypocotyl | Cotyledon |
|---|---|---|---|
| Treatment | (cm) | Length (cm) | Length (cm) |
| 1 | 0.93 | 1.29 | 0.57 |
| 2 | 0.84 | 1.35 | 0.58 |
| 3 | 0.69 | 0.80* | 0.61 |
| 4 | 0.83 | 0.80* | 0.56 |
| 5 | 1.20 | 1.29 | 0.59 |
| Untreated Seed | 1.02 | 1.21 | 0.68 |

*indicates significant differences.

TABLE 4
Normalcy and Malformed Embryos at Day 24 After Dissection

| | | Malformed |
|---|---|---|
| Treatment | Normalcy (%) | Embryos (%) |
| 1 | 100 | 0.0 |
| 2 | 97.1 | 2.9 |
| 3 | 40.0* | 54.1* |
| 4 | 34.3* | 57.1* |
| 5 | 97.1 | 0.0 |
| Untreated Seed | 94.3 | 5.7 |

*indicates significant differences.

### Example 5

This Example compares the use of hydrogen peroxide and a disinfectant containing o-phenylphenol and o-benzyl-p-chlorophenol in disinfection treatments for loblolly pine seed on contamination and germination of dissected embryos.

Methods: The first treatment was identical to the first treatment (hydrogen peroxide) described in EXAMPLE 4, and the second treatment was identical to the fifth treatment (Amphyll) described in EXAMPLE 4. Seed coats were cracked and removed, and the de-coated seeds were placed in sterile glass Petri plates (one plate/treatment) containing wet filter paper until dissection. Dissection was performed in quadrant Petri plates. A maximum of four seed were dissected per quadrant. Embryos were placed on medium 185 containing 50 g/l of sucrose and 18 g/l of agar (Table 1). Organ lengths and normalcy were assessed after 24 days on the medium. There were 35 dissected embryos per treatment, with 5 embryos per plate.

Results: There was no contamination of dissected embryos subjected to either of the two treatments, as shown in Table 5. This confirms that the use of Amphyll is as effective as hydrogen peroxide as a disinfecting agent. There was no significant difference in normalcy, radicle lengths, hypocotyl lengths, cotyledon lengths, and epicotyl presence between the two treatments, as shown in Tables 5 and 6.

TABLE 5
Normalcy and Contamination at Day 24 After Dissection

| | | | Dissection |
|---|---|---|---|
| Treatment | Contamination (%) | Normalcy (%) | Damaged (%) |
| 1 | 0 | 85.7 | 11.4 |
| 2 | 0 | 91.4 | 8.6 |
| Untreated Seed | 0 | 91.4 | 5.7 |

TABLE 6
Organ Lengths and Epicotyl Presence at Day 24 After Dissection

| | Radicle | | | |
|---|---|---|---|---|
| | Length | Hypocotyl | Cotyledon | Epicotyl |
| Treatment | (cm) | Length (cm) | Length (cm) | Presence (%) |
| 1 | 3.4 | 1.2 | 0.7 | 65.7 |
| 2 | 3.2 | 1.2 | 0.7 | 62.9 |
| Untreated | 3.4 | 1.2 | 0.7 | 51.4 |
| Seed | | | | |

### Example 6

This Example describes the efficacy of using a disinfectant containing o-phenylphenol and o-benzyl-p-chlorophenol in disinfection treatments for loblolly pine seed on bacterial contamination of seed.

Methods: The following method was used for disinfecting loblolly pine seed:
(1) Seed was soaked in ultra pure water for one hour with agitation;
(2) Seed in tea strainers was soaked in a 10% solution of Liquinox (VWR Scientific) for one hour with agitation;
(3) Seed was rinsed with running ultra pure water until no foam was apparent;
(4) Seed was soaked in 2.5% v/v Amphyll for 15 minutes;
(5) Seed was rinsed four times for 20 minutes each with sterile ultrapure water;
(6) Seed was soaked overnight in sterile ultrapure water at about 4° C. overnight;
(7) One seed each was placed in a tube with sterile saline, and serial dilutions of 1:10, 1:1000, and 1:10,000 in saline were prepared;
(8) Two milliliters from each dilution was combined with 18 milliliters of TSYG agar (Trypticase Soy Broth 30 g/l, Yeast Extract 5 g/l, Glucose 10 g/l, and Agar 15 g/l) and poured into a Petri plate. Petri plates were incubated for 24 hours at 23° C., after which the number of colonies formed were counted.

Control loblolly pine seed was soaked in sterile ultrapure water overnight, then the same dilution and plating process was repeated. Six seeds from each treatment were tested.

Results: There was almost no bacterial contamination of Amphyll-disinfected seed (1 seed resulted in 1 bacterial colony from undiluted saline), compared to between about 30 and 200 bacteria present on control seed, as shown in Table 7. Thus, the use of 2.5% Amphyll is effective as a disinfecting agent for seed.

TABLE 7
Bacterial Contamination of Control and Amphyll-treated Seed

| Amphyll- | | | | | |
|---|---|---|---|---|---|
| Treated | | Colony | | | Colony |
| Seed | Dilution | Count | Control Seed | Dilution | Count |
| 1 | 0 | 0 | 1 | 0 | 36 |
| | 1:10 | 0 | | 1:10 | 7 |
| | 1:1000 | 0 | | 1:1000 | 0 |
| | 1:10,000 | 0 | | 1:10,000 | 0 |
| 2 | 0 | 0 | 2 | 0 | 31 |
| | 1:10 | 0 | | 1:10 | 13 |
| | 1:1000 | 0 | | 1:1000 | 0 |
| | 1:10,000 | 0 | | 1:10,000 | 0 |
| 3 | 0 | 0 | 3 | 0 | 40 |
| | 1:10 | 0 | | 1:10 | 3 |
| | 1:1000 | 0 | | 1:1000 | 0 |
| | 1:10,000 | 0 | | 1:10,000 | 0 |
| 4 | 0 | 0 | 4 | 0 | 66 |
| | 1:10 | 0 | | 1:10 | 2 |
| | 1:1000 | 0 | | 1:1000 | 0 |
| | 1:10,000 | 0 | | 1:10,000 | 0 |
| 5 | 0 | 0 | 5 | 0 | 180 |
| | 1:10 | 0 | | 1:10 | 5 |
| | 1:1000 | 0 | | 1:1000 | 0 |
| | 1:10,000 | 0 | | 1:10,000 | 0 |
| 6 | 0 | 1 | 6 | 0 | 214 |
| | 1:10 | 0 | | 1:10 | 2 |
| | 1:1000 | 0 | | 1:1000 | 0 |
| | 1:10,000 | 0 | | 1:10,000 | 0 |

### Example 7

This Example describes another exemplary method of the invention for disinfecting seed.

Methods: The following method was used for disinfecting seed:
(1) Douglas-fir seed was soaked in ultra pure water for one hour;
(2) The seed was soaked in a 10% solution of Liquinox (VWR Scientific) for one hour;
(3) The seed was rinsed with water until no foam was apparent;
(4) The seed was soaked in 50% Ultra-Clorox brand bleach (6.00% sodium hypochlorite) for 15 minutes;
(5) The seed was rinsed three times for 10 minutes each with sterile ultrapure water;
(6) The seed was soaked in 50% Clorox for 15 minutes;
(7) The seed was rinsed four times for 20 minutes each with sterile ultrapure water; and
(8) The seed was soaked overnight in sterile ultrapure water 5° C.

While the preferred embodiment of the invention has been illustrated and described, it will be appreciated that various changes can be made therein without departing from the spirit and scope of the invention.

1. A method for disinfecting seed, comprising the step of contacting seed with an effective concentration of a disinfecting agent comprising at least one of o-phenylphenol and o-benzyl-p-chlorophenol.

2. The method of claim 1, wherein the disinfecting agent comprises o-phenylphenol.

3. The method of claim 2, wherein the concentration of o-phenylphenol is between about 0.1% and about 0.7%.

4. The method of claim 1, wherein the disinfecting agent comprises o-benzyl-p-chlorophenol.

5. The method of claim 4, wherein the concentration of o-benzyl-p-chlorophenol is between about 0.05% and about 0.5%.

6. The method of claim 1, wherein the disinfecting agent comprises o-phenylphenol and o-benzyl-p-chlorophenol.

7. The method of claim 1, wherein the seeds are contacted with the disinfecting agent for between about 5 minutes and about 30 minutes.

8. The method of claim 1, wherein the seeds are conifer seeds.

9. The method of claim 8, wherein the conifer seeds are loblolly pine seeds.

10. The method of claim 8, wherein the conifer seeds are Douglas-fir seeds.

11. A method for obtaining a sterile conifer embryo for somatic embryogenesis, comprising the steps of:
(a) contacting a conifer seed with an effective concentration of a disinfecting agent comprising at least one of o-phenylphenol and o-benzyl-p-chlorophenol; and
(b) removing an embryo from the contacted conifer seed under sterile conditions.

12. The method of claim 11 further comprising the step of culturing the embryo in an induction medium to initiate the formation of embryogenic cells.
